(19) United States
(12) Patent Application Publication
(10) Pub. No.: US 2024/0053316 A1
(43) Pub. Date: Feb. 15, 2024

(54) SYSTEM AND METHOD FOR MONITORING AIR QUALITY

(71) Applicant: The STG Group, LLC, Mobile, AL (US)

(72) Inventor: Sheila V. Slaton, Mobile, AL (US)

(73) Assignee: The STG Group, LLC, Mobile, AL (US)

(21) Appl. No.: 18/233,278

(22) Filed: Aug. 11, 2023

Related U.S. Application Data
(60) Provisional application No. 63/397,791, filed on Aug. 12, 2022.

(51) Int. Cl. G01N 33/00; H02J 50/10; G01N 35/00

(52) U.S. Cl. CPC G01N 33/0063; H02J 50/10; G01N 33/0006; G01N 35/00722; G01N 2035/0091; G01N 2035/009

(57) ABSTRACT

A system and method for monitoring air quality while providing charging capabilities to electronic devices is provided. The system generally comprises a charging device, at least one sensor, output device, processor, processor operably connected to the at least one sensor and output device, and non-transitory computer-readable medium coupled to the processor and having instructions stored thereon. Other embodiments may further comprise a computing device having a user interface, which may be used by a user to input user data into the system as well as view environmental data measured by the system. The system is designed to collect environmental data via the at least one sensor and determine whether air quality has become hazardous to the health of a user. In particular, the system is designed to monitor for hazardous gasses, such as carbon monoxide and ozone, and particulate matter, such as lead and arsenic.

### FIELD OF THE DISCLOSURE

The subject matter of the present disclosure refers generally to a portable charging device configured to determine air quality.

### BACKGROUND

Having clean air to breath is necessary for good health. Though many are aware that air quality can have devastating impacts on one's health, there is an alarming amount of illness around the world due to poor air quality. For instance, poor air quality is responsible for more than 100,000 premature deaths in the United States each year, and costs due to air pollution-related illness are estimated at $150 billion per year. In particular, more than 50,000 people in the US visit the emergency room each year due to accidental CO poisoning, and at least 730 people die from said CO poisoning. As such, it is important to ensure that air quality within one's living areas is within a healthy range. However, there is currently no convenient means for one to monitor air quality in their surrounding environment when on the go, which is particularly concerning since air quality can change drastically over distances and within different indoor areas.

Though measures introduced by the EPA have attempted (and in some ways succeeded) in increasing air quality within the US, there are still air quality issues present. Recently, it has come to the scientific community's attention that microplastics are in the air we breath. Further, the scientific community keeps discovering new ways in which air pollution is detrimental to health. For instance, it was recently discovered that smog damages prenatal and young brains, meaning pollution is likely damaging young children in other ways we currently do not understand. Further, it is becoming increasingly apparent that environmental issues caused by global warming are creating previously unforeseen air quality concerns that will have to be monitored. For instance, certain lakes, such as the Great Salt Lake, contain high levels of arsenic. As these lakes dry up, windstorms can carry this arsenic into the lungs of nearby residents, resulting in hospitalization or even death. Furthermore, certain types of molds produce toxic fumes known to cause serious health problems when people are exposed, and as more housing is constructed it is reasonable to assume there will be increases in the number of cases of toxic mold in residential buildings. Due to all of these issues that can contribute to reduced air quality, it is more important than ever to monitor it to ensure the health of individuals.

Unfortunately, most people cannot always carry traditional air quality detection devices on their person. Current air quality measurement devices are often separate computing devices or are configured to be immobile and measure air quality within a designated area. Additionally, the average person has enough electronic devices to keep up with on a daily basis as is. For instance, many people now have a smart phone and at least one other smart device on their person at all times. This makes it difficult to balance the need for more air quality monitoring with the inconvenience of carrying around yet another electronic device. This is particularly true now that many people have become accustomed to a smart phone serving so many functions at one time. By integrating air quality detection devices with components of modern smart devices, one can increase the convenience of owning an air quality detection device and increase one's awareness of air quality conditions within their home and working environments.

Therefore, there is a need in the art for a portable charging device that monitors air quality in order to prevent negative consequences to individuals' health caused by poor air quality

### SUMMARY

A system and method for monitoring air quality while providing charging capabilities is provided. In one aspect, the system allows a user to monitor air quality in an area in which they have access. In another aspect, the system warns a user when air quality within an area has gone below a minimum threshold. In yet another aspect, the system allows a user to charge an electronics device while monitoring air quality. Generally, the system incorporates an air quality sensor into an electronics device charger to obviate the need for a separate power source for both while keeping a user of the system informed about air quality within an environment.

The system comprises at least one sensor, charger, and output device. Other embodiments may further comprise a computing entity, wireless communication device, processor operably connected to the computing entity and wireless communication device, and non-transitory computer-readable medium coupled to the processor and having instructions stored thereon. The processor is configured to receive environmental data and then use this information to determine when air quality has become hazardous to human health. The computing entity may comprise a user interface that may allow a user to view data of the system and/or cause the system to perform an action via commands input by said user. A database may be used to store environmental data and gathered by the system. Additionally, the system may use data gathered to determine air quality in a geographic area to alert users of potential air quality issues in a geographic region in addition to air quality within a particular building expanse in which the system is deployed.

The system is designed to collect air quality in a building expanse and save said data within profiles so that a user may monitor the conditions within said building expanse. In particular, the system is designed to monitor air for toxic conditions that may cause damage to the physical health of individuals in both the short-term and long-term. The at least one sensor is connected to the charging unit in a way such that it receives power when the charging unit is plugged into a power source. The at least one sensor receives power via the charging unit regardless of whether a computing device of the user is secured to the charging unit and receiving power. The at least one sensor may be configured to measure a variety of types of environmental data and transmit that data to the processor. Types of sensors that may be connected to the data aggregator or the computing entity may include, but are not limited to, gas, thermometer, hygrometer, particulate matter sensor, or any combination thereof.

Environmental data of the system may be saved within user profiles, which may be viewed within the user interface of the system. The system may compare the environmental data to attribute thresholds of the system, wherein said attribute threshold is the maximum/minimum value a particular category of condition data may exist before the system is required to take an action, such as send a computer readable signal to a user's computing device to warn of hazardous air quality. Attribute thresholds may be automatically generated by the system or input by a user. A computing entity may be implemented in a number of different forms, including, but not limited to, servers 110, multipurpose computers, mobile computers, etc. Additionally, a computing entity may be made up of a single computer or multiple computers working together over a network, which may communicate via a wired or wireless connection.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 is a diagram illustrating a system embodying features consistent with the principles of the present disclosure.

FIG. 2 is a diagram illustrating a system embodying features consistent with the principles of the present disclosure.

FIG. 3 is a diagram illustrating a user interface of a system embodying features consistent with the principles of the present disclosure.

FIG. 4 is a diagram illustrating the manner in which individual access to data may be granted or limited based on permission levels.

FIG. 5 is a diagram illustrating a system embodying features consistent with the principles of the present disclosure.

FIG. 6 is a diagram illustrating a system embodying features consistent with the principles of the present disclosure.

FIG. 7 is a diagram illustrating a system embodying features consistent with the principles of the present disclosure.

FIG. 8 is a flow chart illustrating certain method steps of a method embodying features consistent with the principles of the present disclosure.

FIG. 9 is a flow chart illustrating certain method steps of a method embodying features consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, steps, etc. are optionally present. For example, a system “comprising” components A, B, and C can contain only components A, B, and C, or can contain not only components A, B, and C, but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). As will be evident from the disclosure provided below, the present invention satisfies the need for a system designed to measure attributes of local air as well as to determine if said local air has become dangerous for a user in order to alert said user.

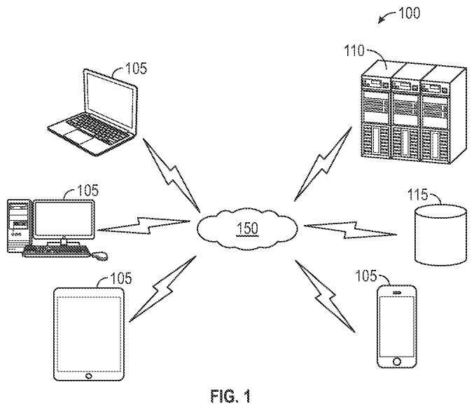

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a streaming device, a “smart” television, a personal digital assistant (PDA), a laptop, a smart phone, a tablet computer, or another type of computation or communication interface 280. Servers 110 may include devices that access, fetch, aggregate, process, search, provide, and/or maintain documents. Although FIG. 1 depicts a preferred embodiment of an environment 100 for the system 400, in other implementations, the environment 100 may contain fewer components, different components, differently arranged components, and/or additional components than those depicted in FIG. 1. Alternatively, or additionally, one or more components of the environment 100 may perform one or more other tasks described as being performed by one or more other components of the environment 100.

As depicted in FIG. 1, one embodiment of the system 400 may comprise a server 110. Although shown as a single server 110 in FIG. 1, a server 110 may, in some implementations, be implemented as multiple devices interlinked together via the network 150, wherein the devices may be distributed over a large geographic area and performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server 110. Additionally, it is understood that multiple servers 110 may be used to operably connect the processor 220 to the database 115 and/or other content repositories. The processor 220 may be operably connected to the server 110 via wired or wireless connection. Types of servers 110 that may be used by the system 400 include, but are not limited to, search servers, document indexing servers, and web servers, or any combination thereof.

Search servers may include one or more computing entities 200 designed to implement a search engine, such as a documents/records search engine, general webpage search engine, etc. Search servers may, for example, include one or more web servers designed to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, wherein a provided webpage may include a reference to a web server at which the desired information and/or links are located. The references to the web server at which the desired information is located may be included in a frame and/or text box, or as a link to the desired information/document.

Document indexing servers may include one or more devices designed to index documents available through networks 150. Document indexing servers may access other servers 110, such as web servers that host content, to index the content. In some implementations, document indexing servers may index documents/records stored by other servers 110 connected to the network 150. Document indexing servers may, for example, store and index content, information, and documents relating to user accounts and user-generated content. Web servers may include servers 110 that provide webpages to clients 105. For instance, the webpages may be HTML-based webpages. A web server may host one or more websites. As used herein, a website may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database 115 and DBMS.

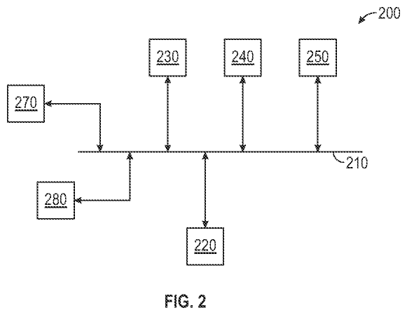

FIG. 2 is an exemplary diagram of a client 105, server 110, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280 (such as wired or wireless communication device). The bus 210 may be defined as one or more conductors that permit communication among the components of the computing entity 200. The processor 220 may be defined as logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

The bus 210 may comprise a high-speed interface 308 and/or a low-speed interface 312 that connects the various components together in a way such they may communicate with one another. A high-speed interface 308 manages bandwidth-intensive operations for computing device 300, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, applications run by a computing entity 200, and wireless communication by a communication interface 280 of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, smart televisions, streaming devices, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, smart televisions, streaming devices, or any other similar device.

Memory 304 stores information within the computing device 300. In some preferred embodiments, memory 304 may include one or more volatile memory units. In another preferred embodiment, memory 304 may include one or more non-volatile memory units. Memory 304 may also include another form of computer-readable medium, such as a magnetic, solid state, or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a controller, scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a streaming device may transfer data to a smartphone, wherein the smartphone may use that data in a manner separate from the streaming device.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

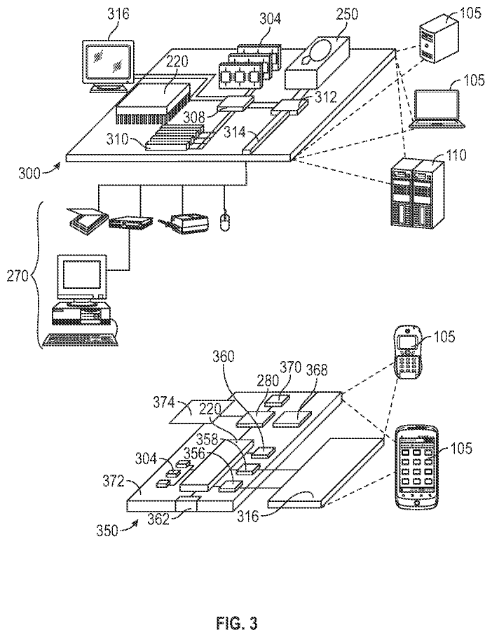

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases 115, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer-readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For example, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System (EMS), Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0), and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation- and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The system 400 may also comprise a power supply. The power supply may be any source of power that provides the system 400 with power. For instance, the power supply may be a stationary power outlet that supplies power via a cable extending from the stationary power outlet to the system 400. For instance, the power supply may be a battery that stores power within and feeds said power directly to the system 400. The system 400 may also comprise of multiple power supplies that may provide power to the system 400 in different circumstances. For instance, the system 400 may be directly plugged into a stationary power outlet, which may provide power to the system 400 so long as it remains in one place. However, the system 400 may also be connected to a backup battery so that the system 400 may receive power even when it is not connected to a stationary power outlet or if the stationary power outlet ceases to provide power to the system 400.

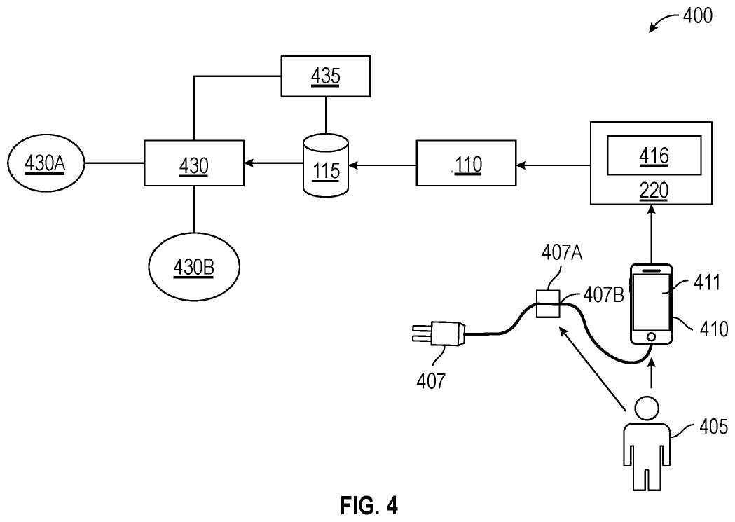

FIGS. 4-9 illustrate preferred embodiments of a system 400 and methods for checking the quality of local air and alerting a user 405 when the air quality of the local air has degraded to the point that said local air has become dangerous to breath. As illustrated in FIG. 4, the system 400 of the present disclosure generally comprises a charging device 407, at least one sensor 407A, output device 407B, processor 220 operably connected to the at least one sensor 407A and output device 407B, and non-transitory computer-readable medium 416 coupled to the processor 220 and having instructions stored thereon. In one embodiment, the system 400 may further comprise a computing entity 200 having a user interface 411 that may be used to control various features of the system 400. In another preferred embodiment, the system 400 may further comprise a database 115 operably connected to the computing entity 200 and used store the various data of the system 400. The database 115 may be populated as the at least one sensors 407A of the system 400 collect environmental data 430B, which may be uploaded directly to the database 115 by the processor 220. Alternatively, the database 115 may be populated by users 405 who inputs data into the user interface 411 of the computing entity 200 that is subsequently transferred to the database 115. In yet another preferred embodiment, a server 110 may be operably connected to the database 115 and processor 220, facilitating the transfer of information between the processor 220 and database 115. The various components of the system 400 of the present disclosure may be operably connected to one another via a wired or wireless connection via a communication device.

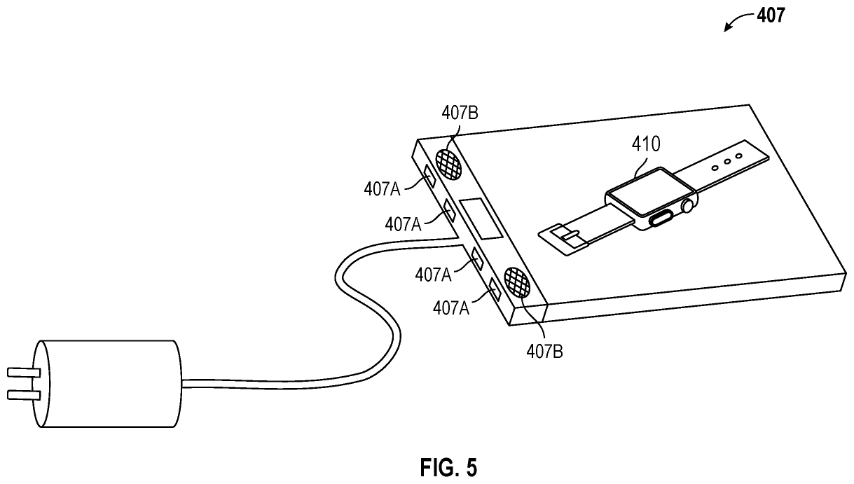

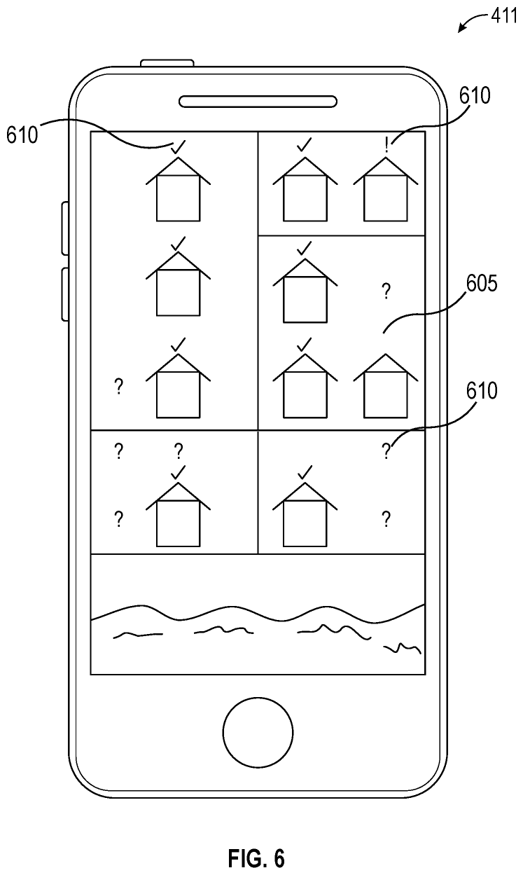

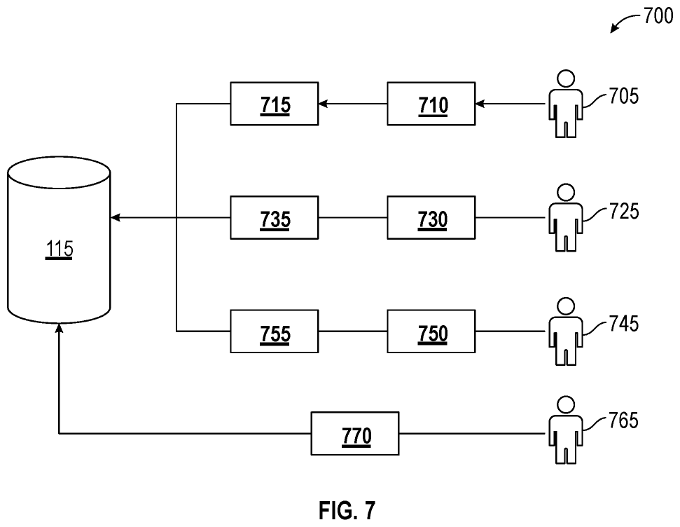

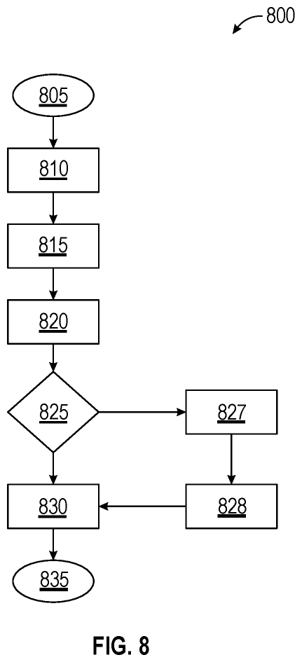

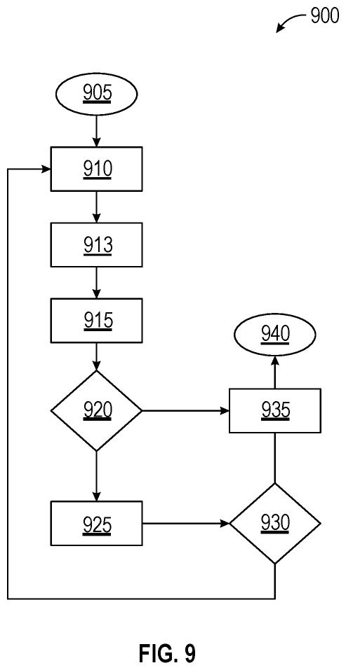

FIG. 4 illustrates a system 400 configured to monitor air quality and alert users 405 if local air has become dangerous to breath. FIG. 5 illustrates a perspective view of a charging device 407 having at least one sensor 407A configured to monitor air quality. FIG. 6 illustrates a user interface 411 of the computing entity 200, wherein said user interface 411 is configured to display various qualities of the air in which it is monitoring. FIG. 7 illustrates permission levels 700 that may be utilized by the present system 400 for controlling access to user content 715, 735, 755. FIG. 8 illustrates a method for installing a charger having at least one sensor 407A configured to monitor air quality and changing attribute thresholds 435 for environmental data 430B pertaining to said air quality. FIG. 9 illustrates a method for rating air quality based on said attribute thresholds 435 and environmental data 430B and alerting a user 405 when said air quality has dropped to a dangerous level. It is understood that the various method steps associated with the methods of the present disclosure may be carried out as operations by the system 400 shown in FIG. 4.

As illustrated in FIGS. 4 and 5, the charging device 407 is configured to connect an electronic device 410, such as a computing entity 200, to a power source while simultaneously connecting the at least one sensor 407A and output device 407B to said power source. For instance, the charging device 407 may be a wireless charging pad configured to wirelessly charge a plurality of smart watches and smart phones. When attached to the power supply, the charging device 407 will facilitate the transfer of power to the at least one sensor 407A and output device 407B. As such, whenever the charging device 407 is receiving power from the power source, the at least one sensor 407A will be measuring environmental data 430B of local air. Should the environmental data 430B be beyond a specific threshold, a computer readable signal will cause the output device 407B, which is also receiving power from the power source via the charging device 407, to output an alert that will warn users 405 of the system 400 that local air has become dangerous to breath. In some preferred embodiments, the charging device 407 may also facilitate the transfer of power from the power supply to the processor 220 and/or communication device. As such, some preferred embodiments of the system 400 are capable of monitoring local air for dangerous conditions without the need of a computing entity 200.

The charging device 407 may be a portable charging device 407 or a stationary charging device 407. Additionally, the charging device 407 may be configured to charge a single electronic device 410 or multiple charging devices 407. For instance, the charging device 407 may be permanently secured to television, wherein said television is meant to remain in a single place for an extended period of time. For instance, the charging device 407 may comprise a plug connector at a first end and a USB-C type connector at a second end, allowing the charging device 407 to be moved from one compatible power supply to another while simultaneously allowing for the charging device 407 to supply power to electronic devices 410 configured to accept said USB-C connector. In a preferred embodiment, the charging device 407 is a wireless charging device that is configured to wirelessly charge compatible electronic devices 410.

The at least one sensor 407A may be secured within the charging device 407 in a way such that it may measure attributes of the local air and transmit environmental data 430B to the processor 220. An attribute may be defined as a trait of the local air in which the charging device 407 is secured to a power source, and environmental data 430B may be defined as data reflecting the state of a particular attribute of local air at a given time. Types of attributes that may be measured by the system 400, include, but are not limited to, temperature, humidity level, parts per million of a particular gas, sound, vibrations, movement, settling, standing water levels, or any combination thereof. Types of sensors that may be used as an at least one sensor 407A include, but are not limited to, a thermometer, hygrometer, gas sensor (including volatile organic compounds (VOCs)), dust sensor, ionization detector, photoelectric detector, or any combination thereof. For instance, a system 400 comprising an at least one sensor 407A comprising a hygrometer, thermometer, and dust sensor may collect humidity data, temperature data, and particulate data and transmit that data to the processor 220. For instance, a system 400 comprising an at least one sensor 407A comprising a gas sensor, ionization detector, and photoelectric detector may collect gas data, ionization data, and photoelectric data and transmit that data to the processor 220. Therefore, the at least one sensor 407A may measure a variety of types of condition data and transmit that data to the processor 220.

Alternatively, the system 400 may receive environmental data 430B from an at least one sensor 407A connected to a computing entity 200. In an embodiment, types of sensors that may be connected to the computing entity 200 may include, but are not limited to, a thermometer, hygrometer, gas sensor (including volatile organic compounds (VOCs)), dust sensor, ionization detector, photoelectric detector, or any combination thereof. The processor 220 may be operably connected to the computing entity 200 in a way such that the environmental data 430B may be transmitted to the processor 220 from the at least one sensor 407A of the computing entity 200. The processor 220 may then use this information to perform the various functions of the system 400. For instance, a computing entity 200 operably connected to the processor 220 may transmit environmental data 430B received from the at least one sensor 407A to the processor 220, wherein said processor 220 may interpret said data, determine an air quality score, and alert a user 405 as to the air quality score. In another preferred embodiment, an alarm system may be operably connected to the computing entity 200. For instance, a computing entity 200 operably connected to the processor 220 may allow the processor 220 to trigger an alarm system operably connected to the computing entity 200 in order to alert a user 405 of local air has become dangerous to breath.

In another preferred embodiment, the system 400 may further comprise a geolocation device configured to collect environmental data 430B in the form of geospatial data. The geolocation device may be a single component or a component of a larger computing entity 200. In one preferred embodiment, the geolocation device may comprise a plurality of devices working together to obtain a geolocation via triangulation. In a preferred embodiment, the geolocation device is a global positioning system (GPS) sensor. The GPS sensor may measure and transmit geospatial data relevant for determining geolocation. A GPS sensor may be defined as a receiver having an antenna designed to communicate with a navigation satellite system. Geospatial data may be spatial data including, but not limited to, numeric data, vector data, and raster data, or any combination thereof. Numeric data may be statistical data which includes a geographical component or field that can be joined with vector files so the data may be queried and displayed as a layer on a map in a geographic information system (GIS) 605. Vector data may be data that has a spatial component, or X, Y coordinates assigned to it. Vector data may contain sets of points, lines, or polygons that are referenced in a geographic space. Raster data may be data in a .JPG, .TIF, .GIF or other picture file format. For instance, a map scanned in a flatbed scanner may be considered raster data.

In a preferred embodiment, user data 430A of a plurality of users may be displayed via a GIS 605 of the user interface in a way such that a user may review the air quality in a given region. For instance, as illustrated in FIG. 6, user data 430A may be used to populate a map to illustrate air quality in a user's neighborhood. In some preferred embodiments, the system 400 may use geospatial data to determine the user's location and present air quality data via a GIS 605 in a way that allows a user to determine the air quality of their immediate surroundings. Indicia may be used to indicate whether air quality poses no threat to the physical health of a user. For instance, as illustrated in FIG. 6, a checkmark may be used to denote areas in which environmental data 430B indicates that air quality is safe for the physical health of the user whereas an exclamation mark may denote areas in which environmental data 430B indicates that air quality is not safe for the physical health of the user. In other embodiments, indicia 610 in the form of colors may be used to denote areas in which air quality is or is not safe. For instance, green highlights on the GIS 605 may denote areas in which environmental data 430B indicates that air quality is safe for the physical health of the user whereas red highlights on the GIS 605 may denote areas in which environmental data 430B indicates that air quality is not safe for the physical health of the user. In some embodiments, indicia 610 may be used to denote areas of the GIS 605 where there is not enough data to determine air quality. For instance, as illustrated in FIG. 6, a question mark may be used to denote locations not possessing data sufficient to determine air quality and its potential effects of the physical health of the user. Indicia 610 may also be used to represent the various buildings, roads, geographic features, etc. within the GIS, as illustrated in FIG. 6.

Some preferred embodiments of the system 400 may further comprise alarm systems configured to detect air pollutants, including, but not limited to, carbon monoxide, smoke, chlorine, hydrogen sulfide, natural gas, propane, or any combination thereof. In a preferred embodiment, the alarm systems comprise at least one sensor and transmit environmental data 430B to the processor. For instance, a wall mounted smoke alarm within a building may transmit environmental data 430B pertaining to particulate matter to the processor. In some embodiments, the alarm systems may be configured to only transmit a computer readable signal to the processor when a pollutant is above an attribute threshold 435. For instance, a wall mounted carbon monoxide alarm that has detected dangerous levels of carbon monoxide in a room may transmit a computer readable signal to the processor instructing the processor to designate an area as dangerous to a user's physical health due to dangerous levels of carbon monoxide. The environmental data 430B and/or computer readable signal transmitted to the processor may be used to create air quality scores and to populate the GIS of the user interface. For instance, a natural gas alarm mounted in a kitchen having a natural gas stove and range may transmit environmental data 430B pertaining to natural gas levels, wherein the natural gas data may be used by the processor to create an air quality score for the air within the kitchen.

In one preferred embodiment, when the system 400 determines that environmental data 430B has gone beyond an allowable level as set by an attribute threshold 435, the processor 220 may send a computer readable signal to the computing entity 200 of a third-party user, wherein the user interface of the computing entity 200 of the third-party user may instruct the third-party user of a potential emergency. In some preferred embodiments, the third-party user is emergency services, such as fireman or paramedics. In other preferred embodiments, the third-party user is emergency dispatch who may use the system 400 to alert the appropriate emergency services of the emergency and type of emergency. For instance, the system 400 may be used to alert firemen that there is a potential carbon monoxide emergency at a particular address, arming the firemen with the appropriate knowledge needed to not only help any potential victims but also to protect themselves. Depending on the permissions of third-party user, the system 400 may send different data. In a preferred embodiment, the system 400 only sends enough data to the third-party user needed to resolve the potential emergency and protect the emergency service providers.

To determine an air quality score, user data 430A, environmental data 430B, and attribute thresholds 435 are gathered and compiled. Based on this data, the system 400 may generate an air quality score that may describe the quality of air in a given geographic area and/or building, such as inside a structure or in a park. In some embodiments, environmental data 430B collected from multiple at least one sensors of multiple charging devices may be combined to create an air quality score for a larger geographic area. In a preferred embodiment, the air quality score is calculated by the system 400 using qualitative analysis methods. This may be performed by the system 400 by determining the various levels of certain compounds and particulate matter within the air and comparing that data with known levels of those compounds and particulate matter that indicate a detrimental impact to a person's health. For instance, the system 400 may use carbon monoxide data, carbon dioxide data, oxygen data, particulate matter data, and geolocation data to predict a health impact likelihood that air will have a negative impact to a person's health and then transform said health impact likelihood into an air quality score by assigning a value thereto. Certain compounds and/or particulate matter may be more detrimental to physical health than other compounds and/or particulate matter; therefore, certain compounds and/or particulate matter may have a greater effect on air quality score than others. For instance, the system 400 may automatically assign an air quality score of zero to an area when it is determined that carbon monoxide levels are at a dangerous level but may only factor pollen levels into a final air quality score no matter how high pollen levels are determined to be based on pollen data.

In another preferred embodiment, the system 400 may calculate the air quality score using a quantitative data analysis. For instance, the air quality score may comprise a plurality of categories that grade the air quality of an area using attribute thresholds 435 based on different criteria having defined limits. In one preferred embodiment, the system 400 may compare the environmental data 430B to an attribute threshold 435, wherein an attribute threshold 435 places an air quality value on a category if the data within that category falls within a defined range. For instance, air quality within a geographic area and/or building may receive one point towards good air quality in a “particulate matter” category containing multiple types of particulate matter if no type of particulate matter measured by the system 400 is above a particular threshold. For instance, if more than one of temperature, humidity, and oxygen levels are not within a defined attribute threshold 435, the air quality for a given geographic area and/or building may receive zero points towards an air quality score in a “physical activity” category. For instance, if chlorine gas is above an attribute threshold 435 in a “lethal” category, the air quality for a geographic area and/or building may automatically receive an air quality score of zero due to negative points being assigned to the category, wherein the negative points assigned are enough points to cause the score to go to zero.

In yet another preferred embodiment, user data 430A may be used to create personalized air quality scores for a user. This type of analysis may be useful to users of the system 400 who would like more personalized control of how the air quality score is generated so they may better determine how air quality may directly impact their health. For instance, a user with user data 430A that indicates that they suffer from COPD may have lower threshold values for certain types of environmental data 430B known to inflame COPD. As such, a first user with COPD may have a lower air quality score displayed within the user interface of their computing entity 200 than a second user would within a user interface of their computing entity 200 with no known health conditions. For instance, a first user having a first user profile containing user data 430A indicating asthma may automatically assign an air quality of zero when particulate matter reaches a certain threshold, whereas a second user having a second user profile containing user data 430A that does not indicate asthma may only slightly lower the air quality score. Further, the attribute threshold 435 for particulate matter may be significantly lower for the first user profile than for the second user not having user profile.

Types of data that may be saved in the system 400 include, but are not limited to, user data 430A, environmental data 430B, attribute thresholds 435, or any combination thereof. User data 430A may be defined as information that may be used to identify a particular user of the system 400. Environmental data 430B may be defined as data pertaining to attributes of air that is local to the at least one sensor 407A. An attribute threshold 435 may be defined as the maximum/minimum value a particular category of environmental data 430B may measure before triggering a warning within the system 400. In a preferred embodiment, attribute thresholds 435 are stored within user profiles 430. Attribute thresholds 435 may be automatically generated by the system 400 or input by a user 405 via the user interface 411. For instance, the system 400 may be configured to automatically set an attribute threshold 435 for carbon monoxide at a maximum value of 50 ppm over an eight-hour period. If the system 400 determines that carbon monoxide within local air is greater than 50 ppm over an eight-hour period, the system 400 may alert the user 405 that the air quality of local air is too dangerous for human inhalation. For instance, a user 405 may change a condition threshold for air oxygen percentage from a minimum value of 19.5% (oxygen deficient) to a minimum value of 21% (standard composition of oxygen normally in air) via a user interface 411 of the computing entity 200. If the system 400 determines that the percentage of oxygen within local air has dropped below 21%, the system 400 may alert the user 405 that the air quality is dangerous for inhalation based on the modified attribute threshold 435.

In a preferred embodiment, the programming instructions responsible for the operations carried out by the processor 220 are stored on a non-transitory computer-readable medium 416 (“CRM”), which may be coupled to the server 110, as illustrated in FIG. 4. Alternatively, the programming instructions may be stored or included within the processor 220. Examples of non-transitory computer-readable mediums 416 include, but are not limited to, magnetic media such as hard disks, floppy disks, and magnetic tape; optical media such as CD ROM discs and DVDs; magneto-optical media such as optical discs; and hardware devices that are specifically configured to store and perform programming instructions, such as read-only memory (ROM), random access memory (RAM), flash memory, and the like. In some embodiments, the programming instructions may be stored as modules within the non-transitory computer-readable medium 416. The software instructions may be read into memory from another non-transitory computer-readable medium 416 or from another device. Alternatively, hardwired circuitry may be used in place of or in combination with software instructions to implement processes consistent with the principles of the invention. Thus, implementations consistent with the principles of the invention are not limited to any specific combination of hardware circuitry and software.

In an embodiment, the system 400 may further comprise a computing entity 200 operably connected to the processor 220. A computing entity 200 may be implemented in a number of different forms, including, but not limited to, servers 110, multipurpose computers, mobile computers, etc. For instance, a computing entity 200 may be implemented in a multipurpose computer that acts as a personal computer for a user 405, such as a laptop computer. For instance, components from a computing entity 200 may be combined in a way such that a mobile computing entity 200 is created, such as mobile phone. Additionally, a computing entity 200 may be made up of a single computer or multiple computers working together over a network. For instance, a computing entity 200 may be implemented as a single server or as a group of servers working together over and Local Area Network (LAN), such as a rack server system. Computing entity 200s may communicate via a wired or wireless connection. For instance, wireless communication may occur using a Bluetooth, Wi-Fi, or other such wireless communication device.

In a preferred embodiment, as illustrated in FIG. 4, the electronic device is a mobile computing device 350. In an embodiment, computing entity 200 may communicate audibly, meaning computing entities 200 may transmit and receive information via sound waves and covert the sound waves into digital information. For instance, a user 405 may instruct a user interface 411 of a computing entity 200 with their voice to perform a certain action. The processor 220 may convert the sound waves of the user 405 into instructions, which the processor 220 may then carry out. Computing entities 200 may likewise generate audible sound for a user 405, such as through an audio device. Such sound may include sound from voice telephone calls, recorded notes, voice messages, music files, etc. Audible sounds may also include sound generated by applications operating on a computing entity 200. For instance, an application running on a mobile computing device 350 may be configured in a way such that when a certain condition is met the application causes the mobile computing device 350 to output a sound. For instance, an application may be configured in a way such that an alarming sound is emitted via an audio device connected to the computing entity 200 to indicate when local air has been determined to be dangerous. For instance, the processor 220 may receive a signal indicating that local air near the user's geolocation has been deemed dangerous, which may cause the processor 220 to emit a computer readable signal that may cause an output device 407B of a mobile computing device 350 to alert the user 405 of the poor/dangerous air quality near their geolocation. In some embodiments, the audible sounds used to alert a user 405 when local air has been deemed dangerous may differ depending on the attribute of local air causing the poor/dangerous air quality or the proximity of the dangerous local air to the user's geolocation.

As mentioned previously, the computing entity 200 may further comprise a user interface 411. A user interface 411 may be defined as a space where interactions between a user 405 and the system 400 may take place. In a preferred embodiment, the interactions may take place in a way such that a user 405 may control the operations of the system 400. A user interface 411 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, and graphical user interfaces, or any combination thereof. In some embodiments, the user interface 411 may be operably connected to back-end hardware, such as a server 110, and/or software that separately handles permission levels 700 of the various users 405. The system 400 may present data of the user interface 411 to the user 405 via a display operably connected to the processor 220. A display may be defined as an output device 407B that communicates data that may include, but is not limited to, visual, auditory, cutaneous, kinesthetic, olfactory, and gustatory, or any combination thereof.

Information presented via a display may be referred to as a soft copy of the information because the information exists electronically and is presented for a temporary period of time. Information stored on the non-transitory computer-readable medium 416 may be referred to as the hard copy of the information. For instance, a display may present a soft copy of visual information via a liquid crystal display (LCD), wherein the hardcopy of the visual information is stored on a local hard drive. For instance, a display may present a soft copy of audio information via a speaker, wherein the hard copy of the audio information is stored on a flash drive. For instance, a display may present a soft copy of tactile information via a vibration device within the computing entity 200, wherein the hard copy of the tactile information is stored within a database 115. Displays may include, but are not limited to, cathode ray tube monitors, LCD monitors, light emitting diode (LED) monitors, gas plasma monitors, screen readers, speech synthesizers, haptic suits, speakers, and scent generating devices, or any combination thereof.

User data 430A, environmental data 430B, and attribute thresholds 435 are preferably saved in user profiles 430 of the system 400. In a preferred embodiment, the user profile 430 may be saved to the non-transitory computer-readable medium 416. Alternatively, the user profile 430 may be saved to a database 115. A user 405 may manually or automatically update a user profile 430 using a computing entity 200. In a preferred embodiment, the computing entity 200 may comprise a user interface 411, which a user 405 may use to manually or automatically update data within the user profile 430. For instance, environmental data 430B may be automatically uploaded to a user profile 430 when detected by the at least one sensor 407A of the system 400. In another preferred embodiment, the user 405 may manually update data within user profiles 430. This may be done through use of, but not limited to, a keyboard, mouse, voice recognition and/or biometric mechanisms, etc. For instance, a user 405 may use a keypad to manually input user data 430A into a user interface 411 of the computing entity 200. Once a user 405 has manually entered the data, the data may be transferred to a user profile 430 within the non-transitory computer-readable medium 416 and/or database 115 via the processor 220. For instance, a user 405 may manually input attribute thresholds 435 into the user interface 411 of a computing entity 200, which may subsequently be obtained from the computing entity 200 by the database 115 via a processor 220 operably connected to the computing entity 200.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database and DBMS.

As shown in FIG. 4, the database 115 may be configured to store data relating to the measurement and grading of air quality. The processor 220 may be operably connected to the database 115 via wired or wireless connection. In a preferred embodiment, the information, data, and/or content 715, 735, 755 associated with air quality may be stored as environmental data 430B and attribute thresholds 435 within a user profile 430. In a preferred embodiment, the database 115 may be configured to store a plurality of user profiles 430 therein and the various information, data, and/or content 715, 735, 755 tied to or associated with such profiles. The database 115 may be a relational database such that the user data 430A, environmental data 430B, and attribute thresholds 435 within each user profile 430 of the plurality user profiles 430 may be stored, at least in part, in one or more tables. Alternatively, the database 115 may be an object database such that the m user data 430A, environmental data 430B, and attribute thresholds 435 within each user profile 430 of the plurality of user profiles 430 may be stored, at least in part, as objects. In some instances, the database 115 may comprise a relational and/or object database and a server 110 dedicated solely to managing the content 715, 735, 755 assigned to user profiles 430 in the manner disclosed herein. Although the database 115 is represented as a single entity within FIG. 4, it is understood that data, information, and/or content 715, 735, 755 stored within the database 115 or repository, as disclosed herein, may be stored within a plurality of databases 115 without departing from the inventive subject matter disclosed herein.

As illustrated in FIG. 7, the system 400 may also comprise a plurality of permission levels 700 that may allow a user 405 to limit what data within their user profiles 430 they share with another user 405. This data may be collectively known as content 715, 735, 755. To access the content 715, 735, 755 stored within the system 400, users 405 may be required to make a request via a user interface 411. Access to the content 715, 735, 755 within the system 400 may be granted or denied by the processor 220 based on verification of a requesting user's 705, 725, 745 permission level 700. If the requesting user's 705, 725, 745 permission level 700 is sufficient, the processor 220 may provide the requesting user 705, 725, 745 access to content 715, 735, 755 stored within the system 400. Conversely, if the requesting user's 705, 725, 745 permission level 700 is insufficient, the processor 220 may deny the requesting user 705, 725, 745 access to content 715, 735, 755 stored within the system 400. In an embodiment, permission levels 700 may be based on user roles 710, 730, 750 and administrator roles 770, as shown in FIG. 7. User roles 710, 730, 750 allow requesting users 705, 725, 745 to access content 715, 735, 755 that a user 405 has uploaded and/or otherwise obtained through use of the system 400. User roles 710, 730, 750 allow users 405 (or requesting users 705, 725, 745 authorized by the user 405) to access the data tied to their own user profiles 430 within the system 400. Administrator roles 770 allow administrators 465 to access system wide data.

In an embodiment, user roles 710, 730, 750 may be assigned to a user 405 in a way such that a requesting user 705, 725, 745 may view user profiles 430 within a database 115 than contain user data 430A, environmental data 430B, and attribute thresholds 435 via a user interface 411. To access the data within the database 115, a user 405 may make a user request via the user interface 411 to the processor 220. In an embodiment, the processor 220 may grant or deny the request based on the permission level 700 associated with the requesting user 705, 725, 745. Only users 405 having appropriate user roles 710, 730, 750 or administrator roles 770 may access the data within the user profiles 430. For instance, as illustrated in FIG. 7, requesting user 1 705 has permission to view user 1 content 715 and user 2 content 735 whereas requesting user 2 725 only has permission to view user 2 content 735. Alternatively, user content 715, 735, 755 may be restricted in a way such that a user 405 may only view a limited amount of user content 715, 735, 755. For instance, requesting user 3 745 may be granted a permission level 700 that only allows them to view user 3 content 755 related to quality scores of local air but not user 3 content 755 related to the exact geolocation of said local air. In the example illustrated in FIG. 7, an administrator 465 may bestow a new permission level 700 on users 405 so that it may grant them greater permissions or lesser permissions. For instance, an administrator 465 may bestow a greater permission level 700 on other users 405 so that they may view user 3's content 755 and/or any other user's content 715, 735, 755. Therefore, the permission levels 700 of the system 400 may be assigned to users 405 in various ways without departing from the inventive subject matter described herein.

FIG. 8 provides a flow chart 800 illustrating certain, preferred method steps that may be used to carry out the method for using a charging device 407 configured to monitor air quality for dangerous conditions. Step 805 indicates the beginning of the method. During steps 810, a user 405 may obtain a charging device 407 having at least one sensor 407A configured to monitor attributes of local air. The user 405 may connect the charging device 407 to a power source during step 815. Once connected, the user 405 may perform a query during step 820 to determine whether to connect an electronic device 410 to said charging device 407 to facilitate the transfer of power between the power source and electronic device 410 via the charging device 407. Based on the results of the query, the user 405 may take an action during step 825. If the user 405 determines that no electronic device 410 must be connected to the charging device 407, the user 405 may proceed to step 830. If the user 405 determines that an electronic device 410 must be secured to the charging device 407, the user 405 may obtain the electronic device 410 during step 827 and subsequently attach the electronic device 410 to the charging device 407 during step 828, allowing power from the power supply to be transferred to the electronic device 410. Once connected, the user 405 may proceed to terminate method step 830.

The system 400 may monitor local air for dangerous attributes so long as the at least one sensor 407A is receiving power. In some embodiments, the charging device 407, at least one sensor 407A, output device 407B, processor 220, and non-transitory computer-readable medium 416 may act independently of any other device. As such, the system 400 will monitor attributes of the local air and warn the user 405 via the output device 407B connected to the charging device 407 should local air become dangerous to breath. However, a user 405 who leaves the vicinity of the charging device 407 might not be able to receive a warning should the user 405 not be able to perceive the output device 407B. In other embodiments, the at least one sensor 407A and/or processor 220 may be operably connected to a computing entity 200 via a network 150. Systems such as these may warn a user 405 via the computing entity 200 in addition to or in lieu of the output device 407B. Therefore, embodiments comprising a computing entity 200 may be configured to warn users 405 of dangerous attributes of local air so long as the at least one sensor 407A is receiving power from the power supply via the charging device 407 and is able to communicate with the computing entity 200.

In one preferred embodiment, the system 400 may use environmental data 430B to generate an attribute score for each attribute of local air and subsequently compare said attribute score to an attribute threshold 435. The attribute threshold 435 may be preset, set manually within the user interface 411 of the system 400 by a user 405, or may be generated by the system 400 using machine learning techniques. In one embodiment, the system 400 may customize an attribute threshold 435 using environmental data 430B from the at least one sensor 407A and user data 430A pertaining to a user's physical health. For instance, a user 405 suffering from COPD may have different attribute thresholds 435 that are considered acceptable for temperature and humidity when compared to users 405 who have no health restrictions within their user profile 430. For instance, a user 405 having user data 430A indicating that said user 405 is the guardian of an infant may have lower attribute thresholds 435 for fine particulate matter when compared to users 405 not in charge of said infant due to the potential effect particulate matter can have on developing brains. Types of thresholds that may act as an attribute threshold 435 include, but are not limited to, temperature, humidity, ppm gas measurements, particulate matter per cubic cm, current, or any combination thereof. When an attribute score of local air is outside of a predefined limit of an attribute threshold 435, the system 400 may send a computer readable signal to a user 405 to alert said user 405 of the potentially dangerous attribute of the local air.

In other preferred embodiments, the system 400 may perform an air quality check to determine the total quality of local air based on all attribute scores of the local air. In some embodiments, the system 400 may alert a user 405 of poor air quality even when no environmental data 430B indicates that a single attribute is outside of an attribute threshold 435. This could be particularly useful in instances where several attributes of local air are near an attribute threshold 435 but not outside said attribute threshold 435. For instance, the system 400 may evaluate air quality based on the presence of particulate matter, carbon monoxide, and methane as well as the percentage of carbon dioxide and oxygen within the local air. If environmental data 430B indicates that local air comprises at least two of particulate matter, carbon monoxide, and methane within 20% of an attribute threshold 435 while simultaneously having elevated carbon dioxide levels and lower oxygen levels, the system 400 may determine that the air quality is poor and alert the user 405 as to the dangers of the local air.

FIG. 9 provides a flow chart 900 illustrating certain, preferred method steps that may be used to carry out the method of alerting a user 405 of poor air quality. During step 905, the processor 220 may receive environmental data 430B from the at least one sensor 407A. Once the environmental data 430B has been received from the at least one sensor 407A, the processor 220 may compare said environmental data 430B to attribute thresholds 435 during step 910. The processor 220 may then perform a query to determine if the environmental data 430B is outside of an attribute threshold 435 during step 615. Based on the results of the query, the user 405 may take an action during step 620. Attribute thresholds 435 are preferably contained within the non-transitory computer-readable medium 416 of the system 400. However, some preferred embodiments of the system 400 may comprise attribute thresholds 435 within user profiles 430, wherein the attribute thresholds 435 within the user profiles 430 are used in lieu of attribute thresholds 435 stored within the non-transitory computer-readable medium 416. As such, some embodiments may require the retrieval of attribute thresholds from user profiles during step 913 prior to performing query step 915.

If the processor 220 determines that environmental data 430B is outside of an attribute threshold 435, the system 400 may proceed to step 935 wherein the system 400 may transmit a computer readable signal. If the system 400 determines that environmental data 430B is not outside of an attribute threshold 435, the system 400 may proceed to step 925, wherein said processor 220 may perform a query to determine if multiple environmental data 430B are within a predefined range of an attribute threshold 435. Based on the results of the query performed during step 925, the processor 220 may take an action during step 930. If the processor 220 determines that multiple environmental data 430B are not within said predefined range of an attribute threshold 435, the system 400 may return to step 910. If the processor 220 determines that multiple environmental data 430B are within said predefined range of an attribute threshold 435, the system 400 may proceed to step 935. During step 935, the system 400 may send a computer readable signal to at least one of an output device 407B and a computing entity 200, wherein said computer readable signal causes the output device 407B and/or computing entity 200 to perform an action that may warn a user 405 of poor/dangerous air quality. Once the computer readable signal has been sent, the system 400 may proceed to the terminate method step 940.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one input/output device.

These computer programs, which may also be referred to as programs, software, software applications, applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter may be made without departing from the principles and scope of the inventive subject matter.

What is claimed is:

1. A system for monitoring air quality within a building expanse comprising:
a charging device configured to receive power from a power source,
wherein said charging device is removably secured to said power source at a first end,
wherein said charging device is configured to charge an electronic device,
at least one sensor operably connected to said charging device,
wherein said at least one sensor is configured to measure environmental data within a building expanse,
wherein said at least one sensor receives power from said power source when said charging device is removably secured to said power source,
a computing device operably connected to said at least one sensor and having a user interface,
wherein said computing device is configured to receive said environmental data from said at least one sensor,
wherein said user interface is configured to allow a user to view environmental data obtained by said at least one sensor,
wherein said computing device is configured to output an alarm signal when said environmental data is outside of an attribute threshold,
a processor operably connected to said at least one sensor and said computing device,
wherein said processor receives said environmental data from said at least one sensor,
wherein said processor intermittently checks said environmental data against an attribute threshold,
wherein said processor sends a computer readable signal to said computing device when said environmental data is outside of said attribute threshold, and
a non-transitory computer-readable medium coupled to said processor and having instructions stored thereon, which, when executed by said processor, cause said processor to perform operations comprising:
receiving said environmental data from said at least one sensor,
receiving user data pertaining to health of a user,
creating an air quality score based on said environmental data and an attribute threshold,
determining whether said air quality is hazardous to physical health of a user by comparing at least one of said environmental data or said air quality score to user data,
transmitting a computer readable signal to said computing device when said environmental data is hazardous to said physical health of said user.

2. The system of claim 1, further comprising an output device operably connected to said charging device and said at least one sensor,
wherein said output device is configured to receive power from said power source when said power source is removably secured to said power source,
wherein said output device is configured to output an alarm signal when said environmental data is outside of an attribute threshold.

3. The system of claim 2, wherein said processor sends a computer readable signal to said output device when said environmental data is outside of said attribute threshold, wherein said computer readable signal instructs said output device to output said alarm signal.

4. The system of claim 1, further comprising a database operably connected to said processor,
wherein said environmental data and user data transmitted to and from said processor are saved within said database,
wherein said database is configured to receive said environmental data and said user data and store said environmental data and said user data in user profiles.

5. The system of claim 4, further comprising a GIS of said user interface,
wherein indicia of said GIS represents a total air quality in a specific geographic region,
wherein said total air quality is determined using said environmental data that was obtained in said specific geographic region.

6. The system of claim 5, further comprising additional instructions:
creating an air quality score based on said environmental data and an attribute threshold,
determining an indicia to present within said GIS of said user interface based on said air quality score, and
presenting said indicia within said GIS of said user interface.

7. The system of claim 1, wherein said attribute threshold is determined based on said user data.

8. The system of claim 7, wherein said air quality score is changed based on said physical health of said user.

9. The system of claim 1, further comprising a magnet of said charging device, wherein said magnet secures a charging base of said charging device to an electronic device.

10. The system of claim 9, wherein said charging base contains a transmitting induction coil configured to transmit a field of energy to a receiving induction coil of said electronic device, wherein said transmitting induction coil and said receiving induction coil are parallel when said charging base is secured to said electronic device.

11. A system for monitoring air quality within a building expanse comprising:
a charging device configured to receive power from a power source,
wherein said charging device is removably secure to said power source,
at least one sensor operably connected to said charging device,
wherein said at least one sensor is configured to measure environmental data within a building expanse,
wherein said at least one sensor receives power from said power source when said charging device is removably secured to said power source,
a processor operably connected to said at least one sensor,
wherein said processor receives said environmental data from said at least one sensor,
wherein said processor intermittently checks said environmental data against an attribute threshold,
a computing device operably connected to said processor and configured to receive said environmental data,
wherein a user interface of said computing device is configured to present environmental data to a user,
wherein a user interface of said computing device is configured to alert said user when said environmental data indicates that air quality has become hazardous to physical health of said user,
wherein said user interface allows said user to input user data into the system,
wherein said computing device is configured to receive a computer readable signal from said processor,
a database operably connected to said processor,
wherein said environmental data and said user data transmitted to and from said processor are saved within said database,
wherein said database is configured to receive said environmental data and said user data and store said environmental data and user data in user profiles, and
a non-transitory computer-readable medium coupled to said processor and having instructions stored thereon, which, when executed by said processor, cause said processor to perform operations comprising:
receiving said environmental data from said at least one sensor,
creating an air quality score based on said environmental data and said attribute threshold,
determining whether said air quality is hazardous to physical health of said user based on said user data and at least one of said environmental data or said air quality score, and
transmitting a computer readable signal when at least one of said environmental data or said air quality is hazardous to said physical health of said user.

12. The system of claim 11, further comprising an output device operably connected to said charging device and said at least one sensor,
wherein said output device is configured to receive said power from said power source when said power source is secured to said power source,
wherein said output device is configured to output an alarm signal when said environmental data is outside of an attribute threshold.

13. The system of claim 12, wherein said processor sends a computer readable signal to said output device when said environmental data is outside of said attribute threshold, wherein said computer readable signal instructs said output device to output said alarm signal.

14. The system of claim 13, further comprising additional instructions:
determining whether said environmental data is outside of an attribute threshold, and
transmitting a computer readable signal to said computing device when said environmental data is outside of said attribute threshold.

15. The system of claim 11, further comprising a GIS of said user interface,
wherein indicia of said GIS represents a total air quality in a specific geographic region,
wherein said total air quality is determined using said environmental data that was obtained in said specific geographic region.

16. The system of claim 11, further comprising a magnet of said charging device,
wherein said magnet secures a charging base of said charging device to an electronic device.

17. The system of claim 16, wherein said charging base contains a transmitting induction coil configured to transmit a field of energy to a receiving induction coil of said electronic device, wherein said transmitting induction coil and said receiving induction coil are parallel when said charging base is secured to said electronic device.

18. A method for determining if local air quality is hazardous to physical health of a user comprising steps of:
obtaining environmental data from at least one sensor of an air quality charger,
wherein said at least one sensor of said air quality charger measures said environmental data within a building expanse,
wherein said at least one sensor receives power from a power source connected to a charging device of said air quality charger when said charging device is connected to said power source,
creating an air quality score based on said environmental data and an attribute threshold,
wherein said attribute threshold is a maximum/minimum value a particular category of environmental data that said environmental data should measure before a computer readable signal containing instructions to warn said user is transmitted,
determining whether said local air quality is hazardous to physical health of said user based on user data and at least one of said environmental data or said air quality score, and
transmitting a computer readable signal to a computing device of said user when at least one of said environmental data or said local air quality is hazardous to said physical health of said user.

19. The method of claim 18, further comprising additional steps of:
transmitting a computer readable signal to an output device of said air quality charger when at least one of said environmental data or said local air quality is hazardous to said physical health of said user,
wherein said output device is configured to receive said power from said power source when said power source is secured to said power source,
wherein said output device is configured to output an alarm signal when said environmental data is outside of an attribute threshold.

20. The method of claim 18, further comprising additional steps of:
determining indicia to present within a GIS of a user interface of said computing device based on said air quality score, and
presenting said indicia within said GIS of said user interface.
